(19) United States
(12) Patent Application Publication
(10) Pub. No.: US 2025/0205265 A1
(43) Pub. Date: Jun. 26, 2025

(54) ASPERULOSIDE AS AN AGENT FOR IMPROVING VASCULAR AND METABOLIC FUNCTION IN OBESITY: ROLE OF NRF2 ACTIVATION

(71) Applicant: The Chinese University of Hong Kong, Hong Kong (CN)

(72) Inventors: Wing Tak, Jack WONG, Hong Kong (CN); Chufeng HE, Hong Kong (CN)

(21) Appl. No.: 18/986,220

(22) Filed: Dec. 18, 2024

Related U.S. Application Data
(60) Provisional application No. 63/613,949, filed on Dec. 22, 2023.

(51) Int. Cl. A61K 31/7048; A61K 45/06; A61P 9/00

(52) U.S. Cl. CPC A61K 31/7048; A61K 45/06; A61P 9/00

(57) ABSTRACT

The subject invention pertains to novel methods of using Asperuloside to activate the nuclear factor erythroid 2-related factor/heme oxygenase-1 (Nrf2/HO-1) pathway in a subject. More specifically, the subject invention reverses the impairments of endothelium-dependent relaxations for use in treating endothelial dysfunction in subjects with obesity.

### CROSS-REFERENCE TO RELATED APPLICATION

The present application claims the benefit of U.S. Provisional Application Ser. No. 63/613,949, filed Dec. 22, 2023, the disclosure of which is incorporated herein by reference in its entirety.

### SEQUENCE LISTING

The Sequence Listing for this application is labeled “CUHK-236XC1-SeqList-19Nov24.xml” which was created on Nov. 19, 2024 and is 4,479 bytes. The entire contents of the sequence listing is incorporated herein by reference in its entirety.

### BACKGROUND OF THE INVENTION

Worldwide, more than one billion people have obesity. Obesity is a disease that impacts most body systems, including heart, liver, kidneys, joints, and reproductive system. Obesity can lead to a range of other diseases, such as type two diabetes, cardiovascular disease, hypertension and stroke, multiple types of cancer, and mental health issues.

Obesity is commonly caused by consuming more calories than expended. The excess energy is stored by the body as fat and contributes to weight gain. Obesity can also be caused by underlying health conditions, such as, for example, underactive thyroid gland (hypothyroidism).

Traditional treatments for obesity include eating a healthy, reduced-calorie diet and regular exercise. There are some medications that can be prescribed to help reduce the amount of fat absorbed during digestion. However, these medications can be effective but have undesirable side effects and are not designed to specifically treat vascular dysfunction. Thus, a novel therapy to target obesity-associated vascular dysfunction is urgently needed.

### BRIEF SUMMARY OF THE INVENTION

To lower the mortality and vascular events in a subject with a body mass index of equal to or greater than 30, thus developing effective clinical therapies for vascular impairments has become an imperative demand. The subject invention provides novel methods to treat vascular dysfunction, including obesity-associated vascular dysfunction and endothelial dysfunction, by administering Asperuloside (ASP) to a subject in need thereof. In certain embodiments, the subject is afflicted by obesity-associated vascular dysfunction, or non-alcoholic fatty liver disease.

In certain embodiments, the administration of ASP to a subject significantly reverses the impairments of endothelium-dependent relaxations and remarkedly inhibits endothelial activation, highlighting the effectiveness of ASP at specifically targeting endothelial dysfunction. In certain embodiments, endothelial activation is decreased by at least 30%, about 50%, about 70%, or preferably about 80%.

In certain embodiments, the administration of an effective amount of ASP activates the Nrf2 signaling pathway. In certain embodiments, ASP directly binds to the Nrf2 protein and increases Nrf2 activity by about 1.5 fold. In certain embodiments, the administration of ASP fully suppresses oxidative stress by activating the nuclear factor erythroid 2-related factor/heme oxygenase-1 (Nrf2/HO-1) pathway in the subject, which is independent of its anti-inflammatory properties, such as inactivating the NF-kB pathway and decreasing IL-1β levels. In certain embodiments, in a condition where the body mass index of equal to or greater than 30, reactive oxygen species (ROS) production is elevated about 2 fold compared to a control group, while ASP treatment can clear all the excessive ROS level and restore ROS production to the level of a control group. In certain embodiments, the ROS level in ASP-treated aortas and endothelial cells is similar to a control group. In certain embodiments, oxidative stress is defined as ROS overproduction and accumulation and can be assessed by dihydroethidium (DHE) staining and/or 2′,7′-dichlorodihydrofluorescein diacetate (H2DCFDA) staining. In certain embodiments, the administration of ASP enhances HO-1 expression by stimulating Nrf2 nuclear translocation, thereby promoting the direct binding of Nrf2 to antioxidant response element (ARE), which is located in the promoter region of HO-1. In certain embodiments, the administration of ASP enhances the expression levels of Nrf2 and HO-1 by about 1.5 fold. In certain embodiments, binding of endothelial Nrf2/antioxidant responsive element (ARE) is dose dependent and increases by at least 2 fold. In preferred embodiments, binding of endothelial Nrf2/antioxidant responsive element (ARE) increases by at least 2.5 fold. In further embodiments, the administration of ASP increases endothelial Nrf2/antioxidant responsive element (ARE) binding by about 50%, 60% and 70% higher than the binding to impaired endothelial cells when 10 μm ASP, 20 μm ASP, or 40 μm ASP are administered, respectively.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H show the effect of ASP on obesity-associated endothelial dysfunction. Representative traces demonstrating the beneficial effect of ASP on impaired acetylcholine-induced endothelium-dependent relaxations (EDRs) in high-fat diet (HFD)-fed mice (FIG. 1A). FIG. 1B summarizes results showing that ASP prevents HFD-induced impairment of EDRs, while endothelium-independent relaxations in response to sodium nitroprusside (SNP) (FIG. 1C) remain similar among different groups. DHE staining demonstrates that ASP suppresses elevated ROS generation in obese mice aortic sections (FIGS. 1D-1E). Real-time PCR analysis (FIG. 1F) reveals that ASP decreases elevated expression of VCAM-1 and ICAM-1 mRNA in the aortas of obese mice. Real-time PCR analysis reveals that ASP increases mRNA expression level of Nrf2 (FIG. 1G) and HO-1 (FIG. 1H) in obese mice aortas. P values were determined using Two-way ANOVA for FIGS. 1B and 1C and One-way ANOVA for FIGS. 1A, 1D, 1E and 1F. **p<0.01 HFD vs. NCD. ##p<0.01 HFD vs. HFD+ASP (FIGS. 1B-1C). *p<0.05 (FIGS. 1A, 1D, 1E and 1F). HFD, high-fat diet; EDRs, endothelium-dependent relaxations; SNP, sodium nitroprusside.

FIGS. 2A-2F show that the inhibition of HO-1 upregulation abolishes the vaso-protective effects of ASP. En-face immunofluorescence staining (FIG. 2A) confirms that ASP fails to increase HO-1 expression in OB-24-treated aortas. ASP prevents IL-1-induced impairment in acetylcholine-induced EDRs (FIG. 2B) but fails to rescue the impairment (FIG. 2C) or decrease ROS generation (FIG. 2D) in HO-1-inhibited aortas. ASP suppresses oxidative stress and inhibits overexpression of VCAM-1 and ICAM-1 in endothelial cells. ASP fails to decrease ROS level (FIG. 2E) or inhibit endothelial activation (FIG. 2F) in HO-1-silencing cells. **p<0.01 control vs. IL-1β. ##p<0.01 IL-1β vs. IL-1β+ASP (FIGS. 2B-2C). *p<0.05 (FIG. 2F).

FIGS. 3A-3G show that ASP activates endothelial Nrf2/HO-1 pathway. En-face Immunofluorescence staining (FIG. 3A) shows that EC-specific knockdown of Nrf2 abolishes the upregulation of HO-1 by ASP treatment. Western blotting (FIG. 3B) shows that ASP-induced upregulation of HO-1 is eliminated in Nrf2-silenced cells. ASP dose-dependently stimulates Nrf2 nuclear translocation, as illustrated by western blotting (FIG. 3C), and increases ARE binding activity, as detected by Luciferase Reporter Assay (FIG. 3D). Immunofluorescence staining (FIG. 3E) and western blotting (FIG. 3F) demonstrate that ML385 co-treatment blocks Nrf2 nuclear translocation stimulated by ASP. Luciferase reporter assay (FIG. 3G) shows that ML385 attenuates the activation of ARE binding by ASP. *p<0.05

FIGS. 4A-4F show that ASP activates Nrf2/ARE binding, thereby elevating HO-1 expression and ultimately improving endothelial function. En-face immunofluorescence staining (FIG. 4A) demonstrates that the inhibition of Nrf2/ARE binding eliminates the endothelial upregulation of HO-1 by ASP treatment. ASP fails to restore IL-1β-mediated downregulation of HO-1 protein (FIG. 4B) and mRNA (FIG. 4C) in ML385-treated cells. ASP prevents IL-1β-impaired EDRs (FIG. 4D), while it fails to reverse impairment (FIG. 4E) or suppress IL-1β-induced ROS overproduction (FIG. 4F) in ML385-treated aortas.

FIG. 5 shows that ASP ameliorates obesity-associated endothelial dysfunction via activating Nrf2/HO-1 pathway. ASP facilitates Nrf2 nuclear translocation and Nrf2/ARE binding, thereby increasing HO-1 expression. Thus, ASP inhibits endothelial activation and suppresses oxidative stress, ultimately alleviating endothelial dysfunction associated with obesity.

FIGS. 6A-6D shows that the specific knockdown of endothelial Nrf2 eliminates vascular protective effects of ASP. Endothelial cell (EC)-specific Nrf2 knockdown mice were generated using the Crispr-cas9 system by injecting EC-enhanced AAV9-mNrf2-sgRNA (AAV-sgNrf2) into EC-specific cas9 overexpression mice via the tail vein. FIG. 6A illustrates representative traces and summarized results showing that ASP rescued IL-1β-impairs EDRs (FIG. 6B) but fails to reverse the impairment (FIG. 6C) in endothelial-cell specific Nrf2 knockdown murine aortas. DHE staining reveals that ASP fails to suppress IL-1β-induced ROS overexpression when endothelial Nrf2 is knocked down (FIG. 6D). Scale bar: 100 μm. Data represent means±SEM of 5 mice. P values were determined using Two-way ANOVA for (B, C) and One-way ANOVA for (D). **p<0.01 control vs. IL-1β. ##p<0.01 IL-1β vs. IL-1β+ASP (B, C). *p<0.05 (D).

FIGS. 7A-7B illustrate CETSA demonstrating that ASP directly binds to the Nrf2 protein. ASP (40 μmol/L) treatment changes the melting behavior of Nrf2 protein in 0.3 ng/mL IL-1β-treated endothelial cells, demonstrating the direct binding of ASP to Nrf2 protein in IL-1β-treated endothelial cells (FIG. 7A). ASP (40 μmol/L) treatment changes the melting behavior of Nrf2 protein in endothelial cells, demonstrating the direct binding of ASP to Nrf2 protein in endothelial cells under basal conditions (FIG. 7B). The Nrf2 protein expression was normalized to total protein. P values were determined using One-way ANOVA. *p<0.05. Data represent means±SEM of 3 experiments.

FIGS. 8A-8D show that ASP promotes Nrf2 nuclear translocation and activates the Nrf2/HO-1 pathway in endothelial cells under basal conditions. Western blotting analysis revealed that 40 μmol/L ASP increases Nrf2 nuclear expressions in endothelial cells. Nuclear Nrf2 expression was normalized to Lamin B1 (FIG. 8A). Western blotting analysis revealed that ASP dose-dependently upregulated HO-1 expression (FIG. 8B). Summarized data of western blotting analysis reveal that ASP dose-dependently upregulates HO-1 expression (FIG. 8C). Real-time PCR analysis shows that ASP dose-dependently increases HO-1 mRNA. mRNA and protein expression were normalized to GAPDH. P values were determined using One-way ANOVA (FIG. 8D). *p<0.05. Data represent means±SEM of 3-6 experiments.

FIGS. 9A-9H show that ASP inhibits lipid accumulation in obese mice livers via activating Nrf2 signaling. Hematoxylin and eosin staining (H&E) (FIG. 9A) and Oil Red O staining (FIG. 9B) demonstrate that ASP attenuates obesity-induced accumulated lipid droplets in obese mice livers. Real-time PCR analysis (FIG. 9C) illustrates that ASP increases the mRNA expression levels of lipolysis-related genes, Npra and Hsl. Moreover, mRNA expression levels of fatty acid synthesis-related genes, including Fas and Acc2, are downregulated in ASP-treated obese mice livers. Oil Red O staining images (FIG. 9D) show that ASP fails to alleviate PA-induced lipid accumulation in Nrf2-inhibited HepG2 cells. Real-time PCR analysis reveals that the PA-stimulated overexpression of mRNA levels of FAS (FIG. 9E), ACC2 (FIG. 9F) and SREBP1C (FIG. 9G) are inhibited by ASP treatment. However, ASP fails to decrease its expression levels in the Nrf2-inhibited HepG2 cells, demonstrating that ASP stimulates DNL through activating Nrf2 signaling. ASP fails to restore palmitic acid-decreased HSL mRNA expression in Nrf2-inhibited HepG2 cells, demonstrating that ASP promotes hepatic lipolysis via Nrf2 activation (FIG. 9H). Data represent means±SEM of 4-6 mice. Scale bar, 50 μm (FIGS. 9A and 9D) and 100 μm (FIG. 9B). P values were determined using One-way ANOVA. *p<0.05.

FIGS. 10A-10E show that ASP restores obesity-induced mitochondrial dysfunction and stimulates mitochondria biogenesis. Summarized results show that ASP treatment increases ATP levels in obese mice livers (FIG. 10A). Real-time PCR analysis shows that the mRNA expressions of Cpt1α (FIG. 10B), Cpt1β (FIG. 10C), mitochondrial complex (FIG. 10D), and Pgc1α (FIG. 10E) are decreased in obese mice livers while ASP treatment reverses these impairments. Data represent means±SEM of 4-6 mice. mRNA expressions were normalized to GAPDH. P values were determined using One-way ANOVA. *p<0.05.

FIGS. 11A-11B show that ASP reverses mitochondrial dysfunction via Nrf2 activation. ASP restores the decreased production of ATP in PA-treated HepG2 cells while inhibition of Nrf2 eliminates the rescue effect of ASP (FIG. 11A). Real-time PCR analysis (FIG. 11B) shows that ASP fails to upregulate the mRNA expression levels of SDHC in PA-treated HepG2 cells when Nrf2 is inhibited, demonstrating that ASP stimulates mitochondrial metabolism via Nrf2 activation. Data represent means±SEM of 3 experiments. mRNA expressions were normalized to GAPDH. P values were determined using One-way ANOVA. *p<0.05.

FIGS. 12A-12B show that ASP attenuates hepatic oxidative stress via activating Nrf2. DHE staining images illustrate that ASP suppresses oxidative stress in obese mice livers. Scale bar: 25 μm (FIG. 12A). DHE staining reveals that ASP treatment fails to attenuate palmitic acid-induced oxidative stress in Nrf2-inhibited HepG2 cells (FIG. 12B). Scale bar: 25 μm. Data represent means±SEM of 3-5 experiments. P values were determined using One-way ANOVA. *p<0.05.

FIGS. 13A-13F show that ASP promotes Nrf2 nuclear translocation and Nrf2/ARE binding, thereby increasing downstream transcription and activating Nrf2 signaling in obese mice livers and palmitic acid-treated HepG2 cells. Immunofluorescence staining (FIG. 13A) reveals that ASP stimulates Nrf2 nuclear translocation in obese mice livers. Western blot analysis (FIG. 13B) shows that the Nrf2 protein level are increased in ASP-treated obese mice livers. Real-time PCR analysis (FIG. 13C) reveals that ASP upregulates gene expressions of Ho-1, Sirt1, Sirt3, Sirt6 and Sod2 in obese mice livers. Immunofluorescence staining (FIG. 13D) reveals that ASP stimulates Nrf2 nuclear translocation in palmitic acid-treated HepG2 cells. Dual-luciferase assay (FIG. 13E) shows that ASP dose-dependently increases the binding activity of ARE in 250 μmol/L PA-treated HepG2 cells for 16 hours, demonstrating that ASP promotes Nrf2/ARE binding in HepG2 cells. Real-time PCR analysis (FIG. 13F) shows that ASP dose-dependently increases mRNA expression of HO-1, SIRT1, SIRT6, and SOD2 in 250 μmol/L PA-treated HepG2 cells for 16 hours. Data represent means±SEM of 3-6 experiments. Scale bar: 25 μm. mRNA expressions were normalized to Gapdh. P values were determined using One-way ANOVA. *p<0.05.

FIGS. 14A-14B show that ASP decreases the weight and sizes of perirenal adipose tissues in obese mice. The ratio of perirenal adipose tissues to body weight was decreased by ASP treatment in obese mice (FIG. 14A). H&E staining (FIG. 14B) shows that ASP treatment remarkably reduces the sizes of adipocytes in obese mice. Scale bar: 25 μm. Data represent means±SEM of 5-6 mice. P values were determined using One-way ANOVA. *p<0.05.

FIGS. 15A-15C show that ASP attenuates de novo lipogenesis (DNL) in perirenal adipose tissues in obese mice. Real-time PCR analysis reveals that ASP decreases mRNA expression levels of Srebp1 (FIG. 15A) and Acc2 (FIG. 15B) in perirenal adipose tissues in obese mice. Acox1 expression was increased in ASP-treated obese mice perirenal adipose tissues (FIG. 15C). Data represent means±SEM of 4-6 mice. P values were determined using One-way ANOVA. *p<0.05.

FIGS. 16A-16C show that ASP suppresses inflammation in obese mice adipose tissues. Real-time PCR analysis reflects that ASP reverses enhanced expression levels of Il-6 (FIG. 16A) and Il-1β (FIG. 16B) in perirenal adipose tissues of obese mice. ASP decreases Tnfα expression in visceral adipose tissues in obese mice (FIG. 16C). Data represent means±SEM of 4-6 mice. P values were determined using One-way ANOVA. *p<0.05.

FIGS. 17A-17B show that ASP attenuates fatty acid uptake of adipose tissues in obese mice. Real-time PCR results shows that ASP inhibited Cd36 upregulation in perirenal adipose tissues (FIG. 17A) and subcutaneous adipose tissues (FIG. 17B). Data represent means±SEM of 4-6 mice. P values were determined using One-way ANOVA. *p<0.05.

FIG. 18 shows that ASP stimulates lipolysis in perirenal adipose tissues in obese mice. Four to five Real-time PCR experiments show that ASP treatment downregulates Nprc expression. Data represent means±SEM of 4 mice. P values were determined using One-way ANOVA. *p<0.05.

FIGS. 19A-19B show that ASP activates the lipolysis process in both subcutaneous and visceral adipose tissues in obese mice. Real-time PCR analysis reveals that ASP upregulates Hsl expression in subcutaneous adipose tissues in obese mice (FIG. 19A). Nprc expression was increased in visceral adipose tissues in obese mice while ASP treatment decreased Nprc expression, indicating that ASP stimulates lipolysis in visceral adipose tissues in obese mice (FIG. 19B). Data represent means±SEM of 4-6 mice. P values were determined using One-way ANOVA. *p<0.05.

FIGS. 20A-20B show that ASP attenuates macrophage infiltration in obese mice adipose tissues. Real-time PCR analysis reveals that ASP suppresses the upregulation of Cd11c (FIG. 20A), Cd11b (FIG. 20B), and F4/80 (FIG. 20C) in visceral adipose tissues of obese mice. ASP reverses the elevated expression of F4/80 in subcutaneous adipose tissues of obese mice (FIG. 20D). Data represent means±SEM of 3-6 mice. P values were determined using One-way ANOVA. *p<0.05.

FIGS. 21A-21B show that ASP stimulates lipolysis and inhibits fatty acid uptake in primary adipocytes. Primary adipocytes were treated with ASP overnight. Real-time PCR analysis shows that ASP dose-dependently decreases mRNA expression levels of Nprc (FIG. 21A) and Cd36 (FIG. 21B) in primary adipocytes. Data represent means±SEM of 4 mice. P values were determined using One-way ANOVA. *p<0.05.

FIGS. 22A-22B show that ASP stimulates Nrf2 activation in adipocytes. Primary adipocytes were treated with 40 μmol/L ASP (FIG. 22A) or different doses of ASP (FIG. 22B) for 24 hours. Immunofluorescence staining (FIG. 22A) shows that ASP stimulates Nrf2 nuclear translocation in adipocytes. Real-time PCR analysis shows that ASP dose-dependently increases mRNA expression of Nrf2 in adipocytes. Scale bar, 50 μm (FIG. 22B). Data represent means±SEM of 4 experiments. P values were determined using One-way ANOVA. *p<0.05.

### BRIEF DESCRIPTION OF THE SEQUENCES

SEQ ID NO: 1: Nrf2 silencing sense siRNA

SEQ ID NO: 2: Nrf2 silencing antisense siRNA

SEQ ID NO: 3: HO-1 silencing sense siRNA

SEQ ID NO: 4: HO-1 silencing antisense siRNA

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, “subject”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; livestock, such as cattle, sheep, pigs, goats and horses; domestic mammals, such as dogs and cats; laboratory animals, including rabbits, mice, rats and guinea pigs. The term does not denote a particular age or gender. Thus, adult, young, and new-born individuals are intended to be covered as well as male and female subjects. In some embodiments, a host tissue is derived from a subject. In some embodiments, the subject is a non-human subject.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

As used herein, the term “treatment” refers to eradicating; reducing; ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 60, 70, 80, 90, 100 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 9 months, 1 year, 1.5 years, 2 years, 2.5 years, 5 years, or more than 10 years. The frequency and duration of administration of multiple doses of the compositions is such as prevent or treat endothelial dysfunction. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of testing for endothelial dysfunction, such as, for example, magnetic resonance imaging or positron emission tomography. In some embodiments of the invention, the method comprises administration of the compounds at several times per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

As used herein, “obese” describes a subject with abnormal or excessive fat accumulation that presents a risk to health with a body mass index of equal to or greater than 30.

As used herein, “Asperuloside” and “ASP” are used interchangeably to refer to compounds with the molecular formula C₁₈H₂₂O₁₁ (see Formula I). ASP is also called rubichloric acid.

### Formula I:

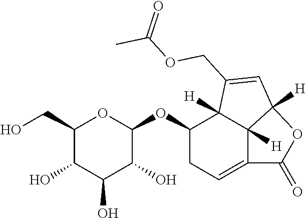

As used herein, “endothelial dysfunction” refers to the alterations in endothelium regulating functions, resulting in imbalanced production of relaxing and contracting factors, pro-coagulant and anticoagulant mediators, or growth-inhibiting and promoting substances, which is characterized by decreased production or bioavailability of nitric oxide (NO).

As used herein, “endothelium-dependent relaxations” refer to the vasodilation that is mediated by the release of an endothelium-derived relaxing factor (EDRF) from vascular endothelium.

As used herein, “endothelial activation” refers to the overexpression of endothelial-surface adhesion molecules, including, for example, VCAM-1, ICAM-1, and endothelial leukocyte adhesion molecule (ELAM).

### Asperuloside Compositions

Provided herein are compounds and compositions thereof for activating the nuclear factor erythroid 2-related factor/heme oxygenase-1 (Nrf2/HO-1) pathway in a subject, wherein the compound is ASP. In certain embodiments, ASP is present in a composition at a concentration of about 1 μg/mL about 100 mg/mL. In certain embodiments, ASP can be administered at a dose of about 1 mg/kg to about 100 mg/kg, about 25 mg/kg to about 75 mg/kg, or about 50 mg/kg.

In certain embodiments, the ASP composition can be administered intraperitoneally, transdermally, and orally, which are adopted by scientists in existing studies.

The composition of the subject invention can also include additives commonly used in medications and other compositions to treat endothelial dysfunction including, for example, aspirin or other nonsteroidal anti-inflammatory compounds, blood pressure medications, including, for example, calcium channel blockers (e.g., Amlodipine, Diltiazem, Felodipine, Isradipine, Nicardipine, Nifedipine, Nisoldipine, Verapamil), cholesterol-lowering drugs, including, for example statins (e.g., Atorvastatin, Fluvastatin, Lovastatin, Pitavastatin, Pravastatin, Rosuvastatin, Simvastatin), or nitrates. In certain embodiments, the composition of the subject invention can also be combined with dietary and lifestyle changes, including for example, eating vegetables, fruits, nuts, whole grains, lean animal protein, fish while eliminating or reducing the consumption of trans fats, saturated fats, red meat, processed meats, refined carbohydrates, alcohol and sweetened beverage, reducing, stress, or eliminating or reducing smoking.

In certain embodiments, the therapeutically effective amount of the composition of the invention can be administered through intraperitoneal administration or by sustained release systems, such as semipermeable matrices of solid hydrophobic polymers containing the compounds of the invention. Administration may be also by way of other carriers or vehicles such as patches, micelles, liposomes, vesicles, implants (e.g., microimplants), synthetic polymers, microspheres, nanoparticles, and the like. In certain embodiments, the compositions may be administered using a nanoparticle to passage the composition through skin.

In certain embodiments, the compositions of the instant invention may be formulated for parenteral administration e.g., by injection, for example, bolus injection, intravenous administration, intraperitoneal administration, or continuous infusion. In addition, the compositions may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

The subject compositions can further comprise one or more pharmaceutically acceptable carriers, and/or excipients, and can be formulated into preparations, for example, semi-solid or liquid forms, such as solutions or injections.

The term “pharmaceutically acceptable” as used herein means compatible with the other ingredients of a pharmaceutical composition and not deleterious to the recipient thereof.

Carriers and/or excipients according to the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaternium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In preferred embodiments, the administration of at least one dose of the composition is repeated at least daily about 7 weeks or longer. In certain embodiments, the repeated administrations of at least one of dose of the composition occurs for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks or longer. In preferred embodiments, the administration of at least one dose of the composition is repeated every about 2 days to about 5 days for about 2 weeks to about 10 weeks.

### Methods of Use

In certain embodiments, the compositions according to the subject invention can be used in methods of increasing the nuclear factor erythroid 2-related factor/heme oxygenase-1 (Nrf2/HO-1) pathway in a subject, wherein a compound or composition thereof targets the Nrf2 protein in a subject. In preferred embodiments, the compound is ASP. In certain embodiments, the compositions according to the subject invention can be used in methods for treating endothelial dysfunction. In certain embodiments, the compositions of the subject invention are administered to a subject in need thereof. The compositions can preferably be administered orally. However, the compositions can be administered using different routes, such as, for example, enteral, intramuscular, intravenous, oral, subcutaneous, sublingual, transdermal, or any combination thereof. Furthermore, ASP or compositions thereof can be also developed as a dietary supplement to improve vascular health and ameliorate vascular complications for subjects, especially for those who are overweight or obese.

In certain embodiments, the subject has obesity-associated vascular dysfunction, or non-alcoholic fatty liver disease. In certain embodiments, the subject is a mammal. In preferred embodiments, the mammal is a human, where preferably the human has a body mass index of equal to or greater than 30. In certain embodiments, the human is overweight.

In certain embodiments, ASP binds directly to the Nrf2 protein. In certain embodiments, ASP binding to Nrf2 increases Nrf2 activity. In certain embodiments, ASP binding to Nrf2 increases Nrf2 activity in endothelial cells. In certain embodiments, ASP binding to Nrf2 increases Nrf2 activity in the liver. In certain embodiments, ASP stimulates Nrf2 nuclear translocation and increases transcription of Nrf2 in the liver. In certain embodiments, ASP increases endothelial-dependent relaxations, suppresses oxidative stress, reduces VCAM-1 expression by about 2 fold and ICAM-1 expression by about 2 fold, and increases both Nrf2 expression by about 1.5 fold and HO-1 expression by about 1.5 fold. In certain embodiments, ASP activates Nrf2/HO-1 signaling by stimulating Nrf2/ARE binding. In certain embodiments, ASP rescues endothelial dysfunction and suppresses vascular oxidative stress associated with obesity and IL-1β via activating the Nrf2/HO-1 pathway. In certain embodiments, the administration of ASP to the subject promotes Nrf2 nuclear translocation and activates the Nrf2/HO-1 pathway, which reverses an impairment of endothelium-dependent relaxation. In further embodiments, ASP stimulates Nrf2 nuclear translocation, wherein the protein level of Nrf2 in the liver increases by about 3 fold and promotes Nrf2 nuclear translocation in palmitic acid-impaired HepG2 cells. Additionally, in embodiments, ASP promotes Nrf2/ARE binding by about 2 fold and Nrf2 nuclear translocation in palmitic acid-impaired HepG2 cells. In certain embodiments, ASP decreases lipid accumulation in the liver from about 50% to 100%. In certain embodiments, ASP stimulates Nrf2 nuclear translocation and dose-dependently increases by at least 1.2 fold Nrf2 mRNA expression in primary adipocytes. In preferred embodiments, ASP stimulates Nrf2 nuclear translocation and dose-dependently increases by at least 1.5 fold Nrf2 mRNA expression in primary adipocytes.

In certain embodiments, ASP fully alleviates mitochondrial dysfunction in the liver by activating hepatic Nrf2 signaling, which promotes Nrf2 nuclear translocation in the liver, thus increasing protein level of Nrf2 by about 3 fold and HO-1 mRNA expression by about 2.5 fold. ASP also increases by at least 2 fold the HO-1 mRNA expression in palmitic acid-impaired HepG2 cells.

In further embodiments, the administration of ASP attenuates lipogenesis, fatty acid uptake in primary adipocytes, inflammation, and macrophage infiltration and promotes lipolysis in perineal adipose tissues. In certain embodiments, perirenal adipose tissues decreases in weight and sizes by about 20%, 25%, 30%, preferably 35%, more preferably 40%, and most preferably 50%. In certain embodiments, perirenal adipose tissues decreases in weight and sizes by an average of about 35%. In some embodiments, ASP stimulates Nrf2 nuclear translocation and dose-dependently increases Nrf2 mRNA expression in primary adipocytes.

In certain embodiments, the administration of ASP alleviates mitochondrial dysfunction in the liver by activating hepatic Nrf2 signaling. In certain embodiments, ASP attenuates oxidative stress in the liver by activating Nrf2 signaling pathway.

In certain embodiments, ASP treatment is not toxic to the subject, including in the heart, liver, and intestine. In certain embodiments, the methods of the subject invention have ameliorative effects of ASP on endothelial dysfunction. In certain embodiments, ASP can improve vascular health for obese subjects. Furthermore, ASP can be an Nrf2 activator to prevent other diseases that are induced by oxidative stress, such as, for example, diabetic neuropathy and non-alcoholic fatty liver disease.

### Materials and Methods

### ASP Treatment in Obese Mice

After induction of obesity in C57BL/6 male mice fed with high fat diet for twelve weeks, they were treated daily with ASP (50 mg/kg) via oral gavage for seven weeks in vivo study. C57BL/6 mice aortae were used for ex vivo study and endothelial cells were used for in vitro study. Vascular function was determined by wire myograph. DHE and MitoSOX staining were used to detect ROS levels. QPCR, aortic en-face immunofluorescence staining, and Western blotting were used to analyze mRNA and protein expressions. Small interfering (si)RNA of Nrf2 and HO-1, inhibitors of Nrf2 (ML385, Cat. No.: HY-100523) and HO-1 (OB-24, HY-118487) were used to elucidate the mechanistic pathway of ASP's action.

### ASP Treatment in Obese Mice

After induction of obesity in C57BL/6 male mice fed with the high-fat diet for twelve weeks, they were treated daily with ASP (50 mg/kg) via oral gavage for seven weeks in vivo study. C57BL/6 mice aortae were used for ex vivo studies. Endothelial cells, HepG2 and primary adipocytes were used for in vitro studies. Vascular function was determined by wire myograph. H&E staining was employed for histological examination. Oil Red O staining was used to determine lipid droplets in tissues and cells. DHE and MitoSOX staining were used to detect ROS levels. QPCR, aortic en-face immunofluorescence staining, and Western blotting were used to analyze mRNA and protein expressions. ARE binding activity was determined by Dual luciferase reporter assay. Small interfering (si)RNA of Nrf2 and HO-1, inhibitors of Nrf2 (ML385, Cat. No.: HY-100523) and HO-1 (OB-24, HY-118487) were used to elucidate the mechanistic pathway of ASP's action.

### Sequences of Small Interfering (si) RNA:

Nrf2 silencing sense:
(SEQ ID NO: 1)
GCAGGACAUGGAUUUGAUUTT
Nrf2 silencing antisense:
(SEQ ID NO: 2)
AAUCAAAUCCAUGUCCUGCTG
HO-1 silencing sense:
(SEQ ID NO: 3)
AGAAGGCUUUAAGCUGGUGAUTT
HO-1 silencing antisense:
(SEQ ID NO: 4)
AUCACCAGCUUAAAGCCUUCUTT

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1-Endothelium-Dependent Relaxations

ASP treatment significantly reversed the impairments of endothelium-dependent relaxations, indicating that ASP specifically targets endothelial dysfunction. Moreover, ASP exhibited remarkable repression of endothelial activation, which was induced by obesity and IL-1β, in aortic tissues and endothelial cells, further confirming the therapeutic effects of ASP on preventing endothelial dysfunction. Strikingly, the underlying mechanism by which ASP protects endothelial function is through the attenuation of oxidative stress via HO-1 upregulation, which is independent of its inhibition of inflammatory responses. Consistent with the findings, results from both ex vivo and in vitro studies, which employed both pharmacological and genetic approaches, provide compelling evidence supporting the indispensability of HO-1 upregulation for the beneficial effects of ASP on mitigating endothelial dysfunction. The data collected from endothelial cell (EC)-specific Nrf2 knockdown mice (Nrf2 is only knockdown in endothelial cells) reflected the reliance of Nrf2 regulation on ASP-elevated HO-1 expression, highlighting that ASP activates endothelial Nrf2/HO-1 pathway. Furthermore, by employing ML385, an inhibitor of Nrf2 binding, it was shown that ASP enhances HO-1 expression by stimulating Nrf2 nuclear translocation and thereby promoting Nrf2/ARE binding. ASP directly binds to Nrf2 protein and thereby increases Nrf2 activity, showing that Nrf2 serves as the receptor for ASP.

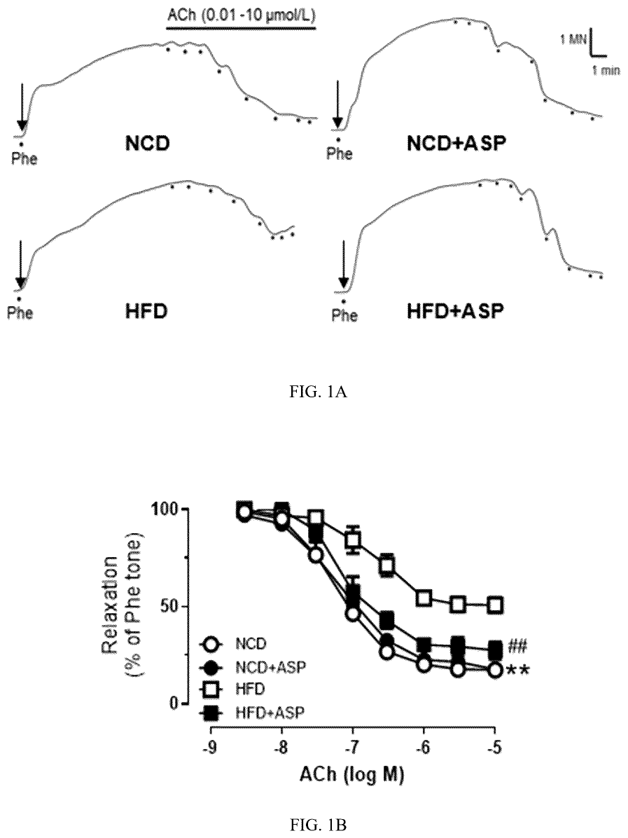

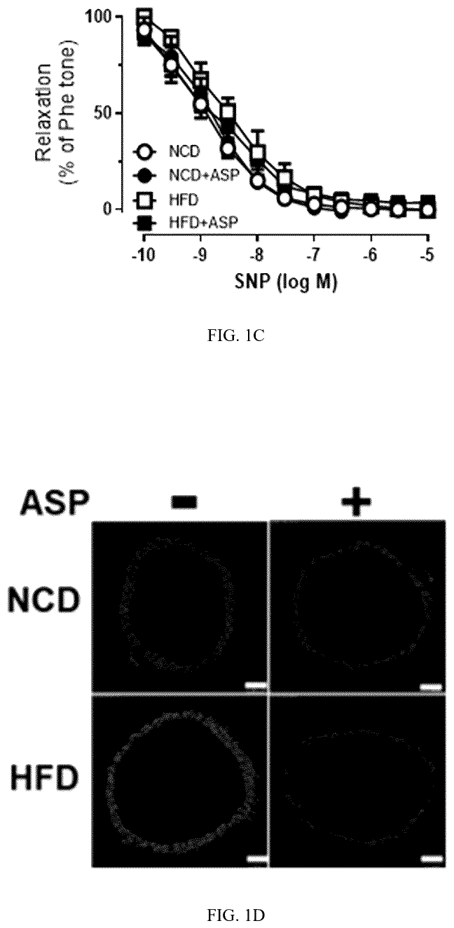

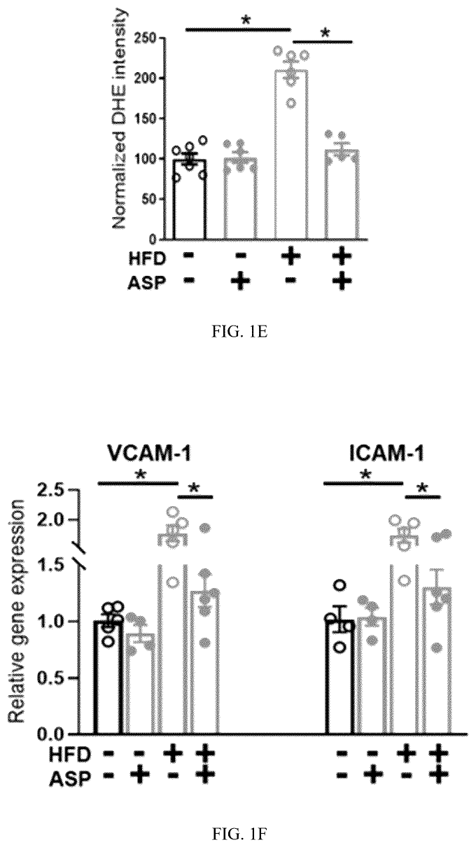

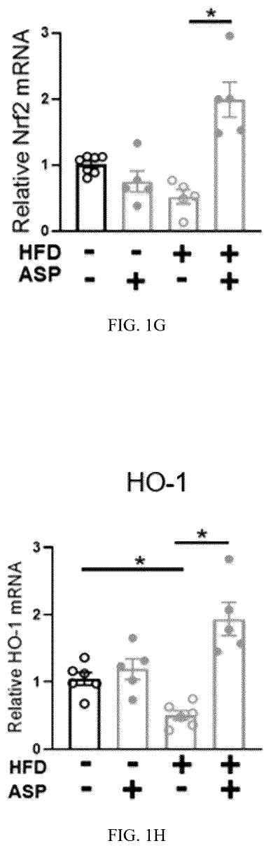

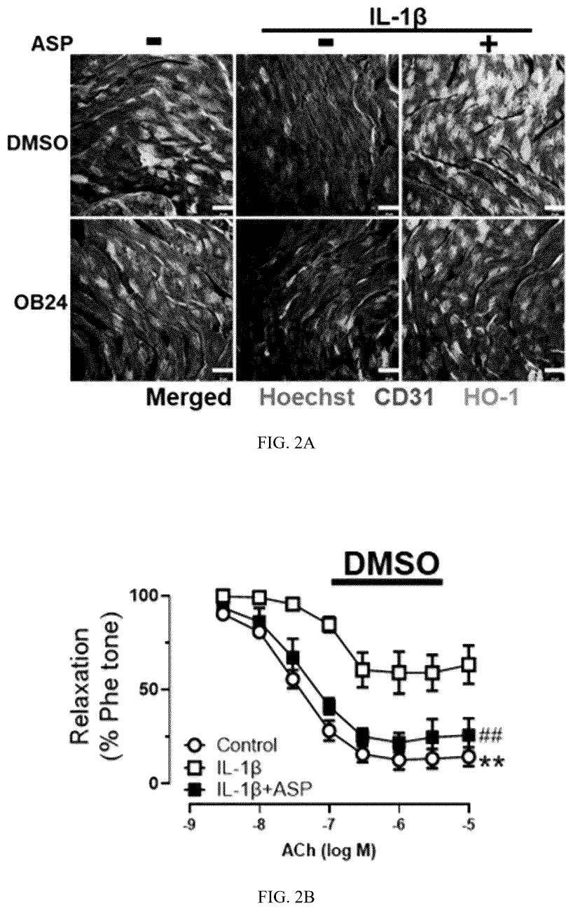

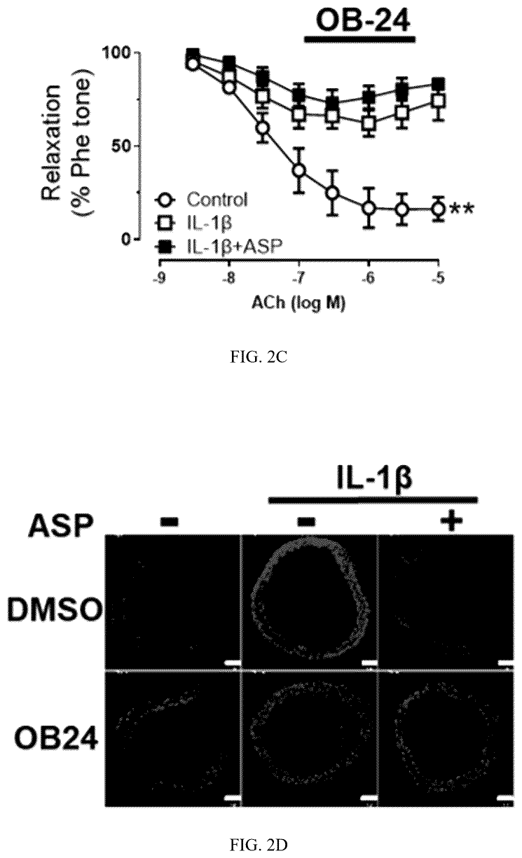

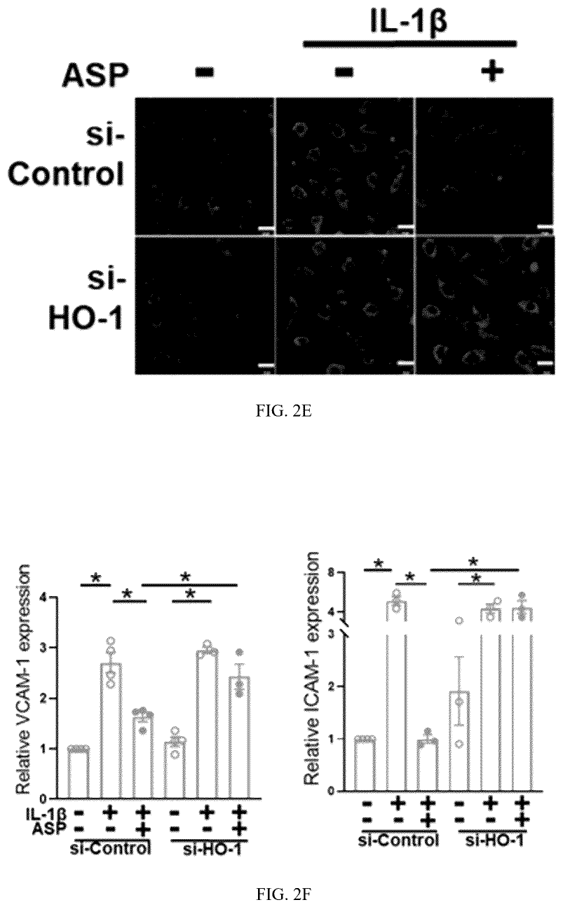

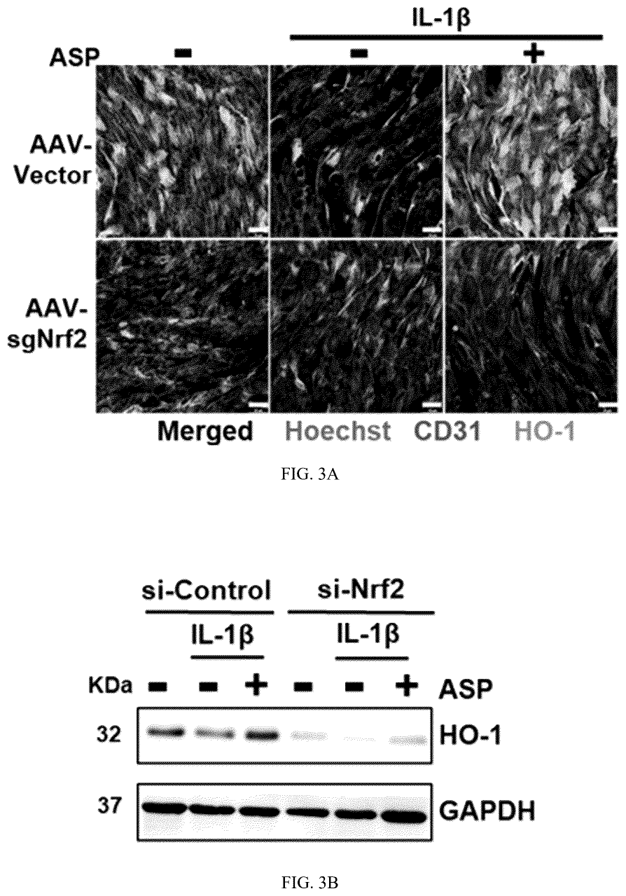

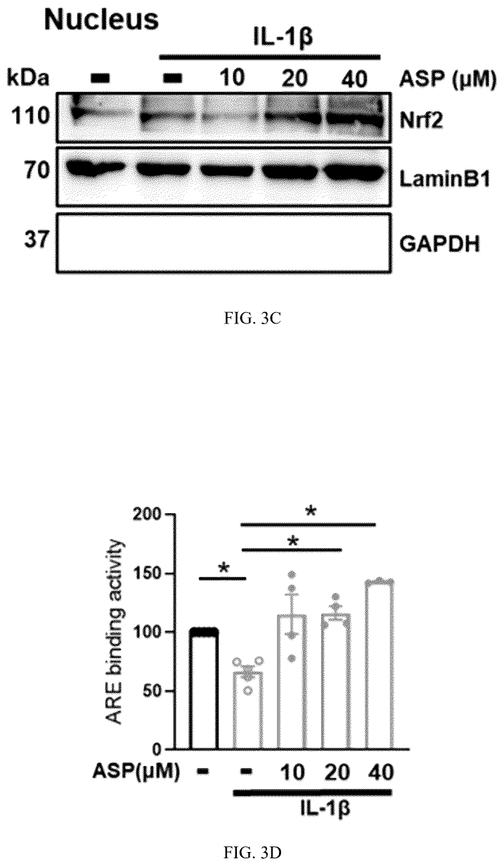

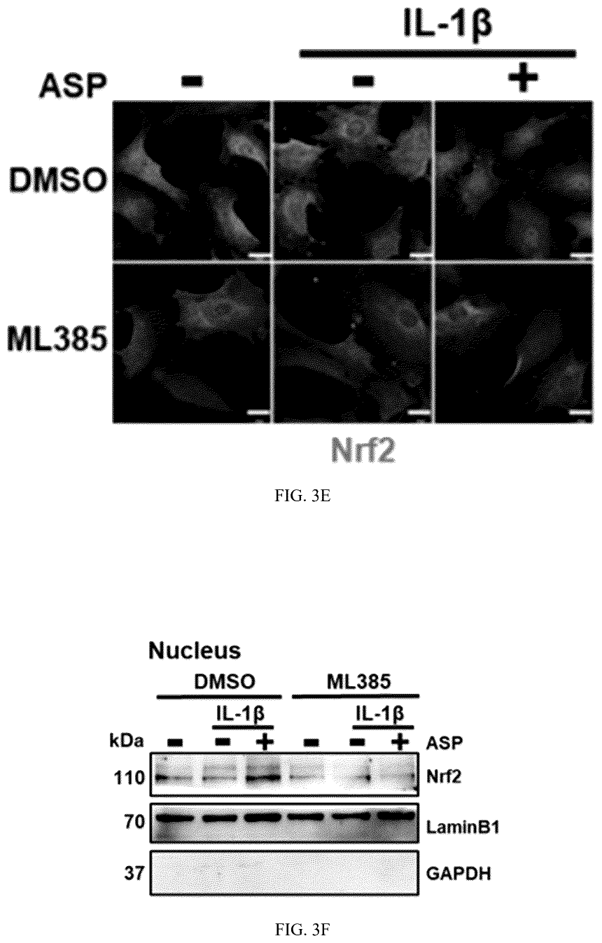

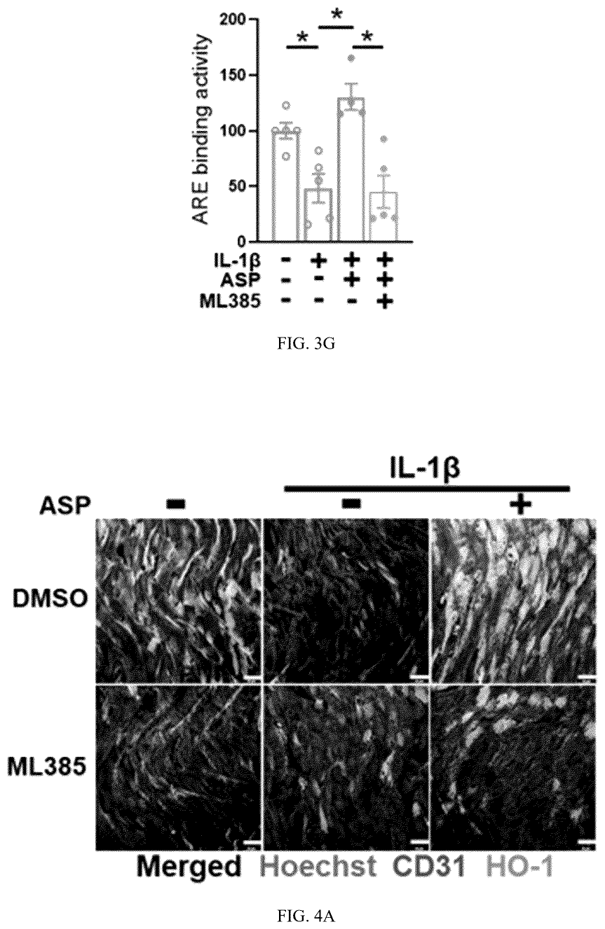

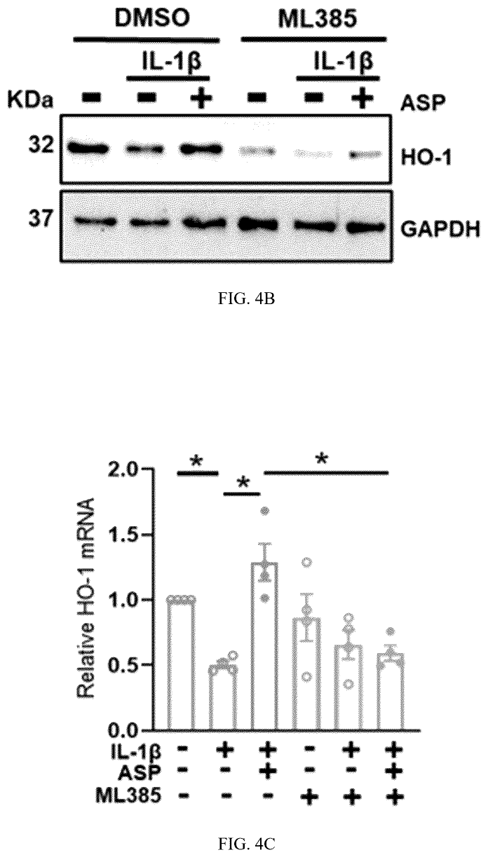

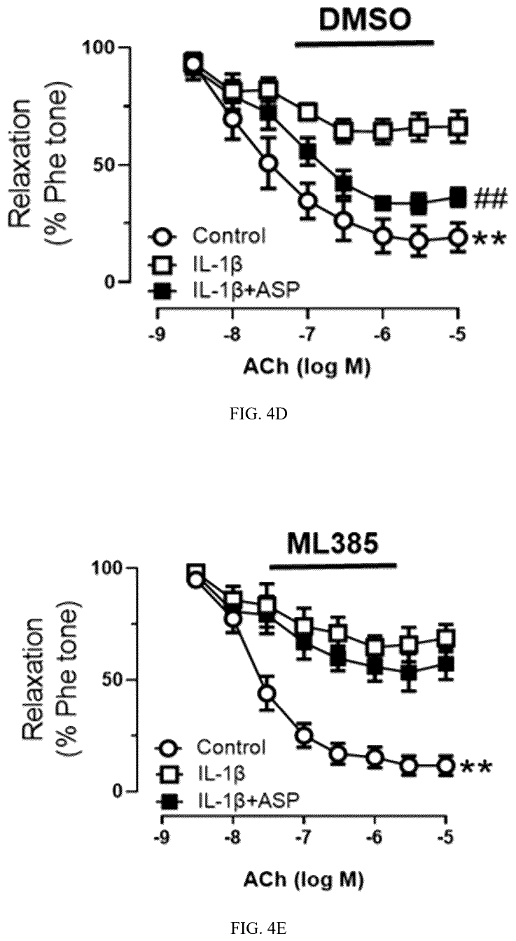

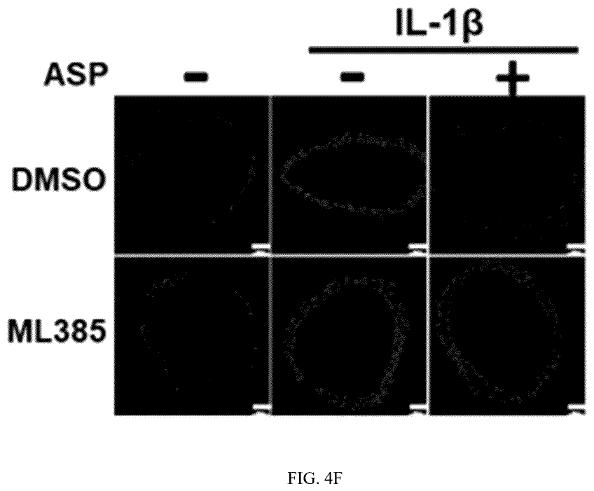

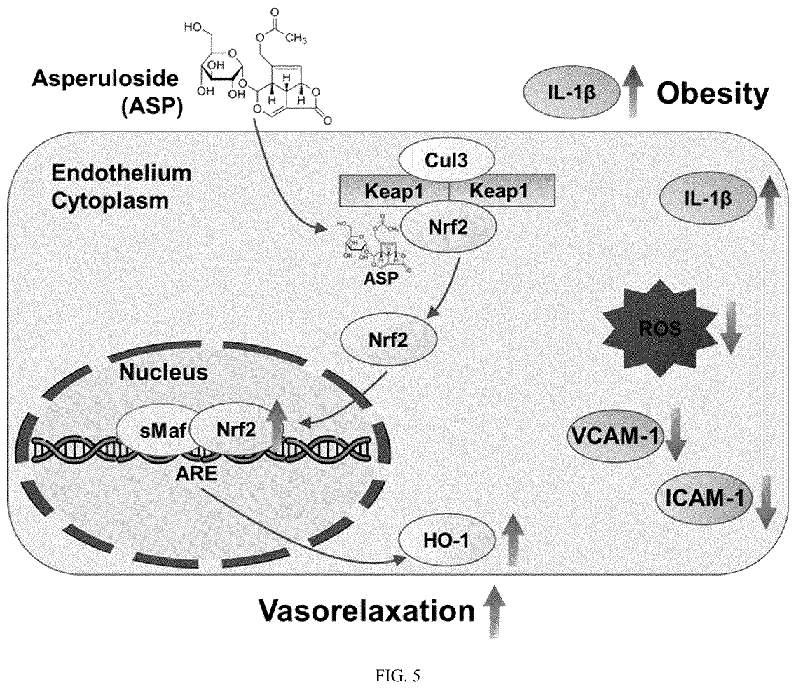

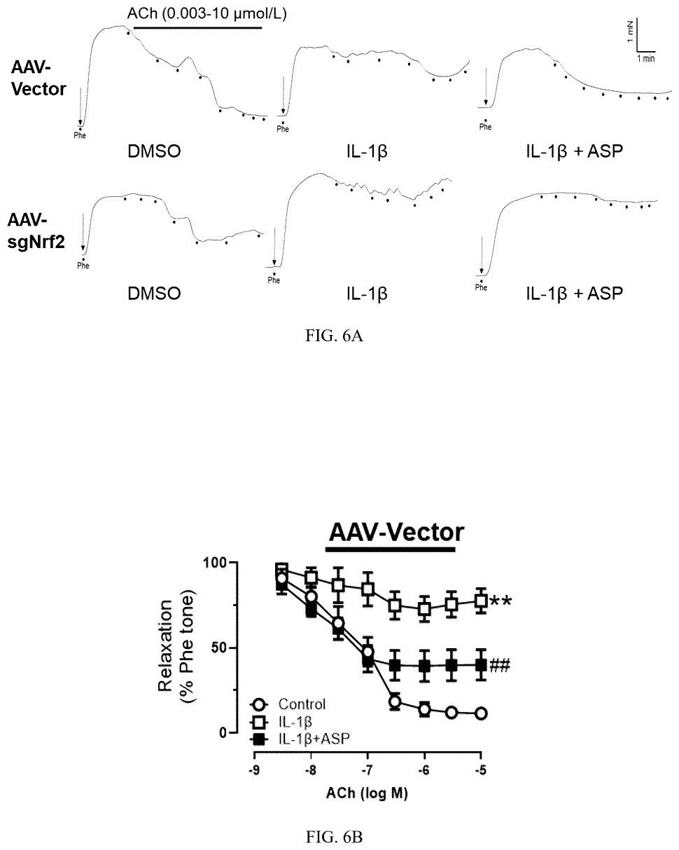

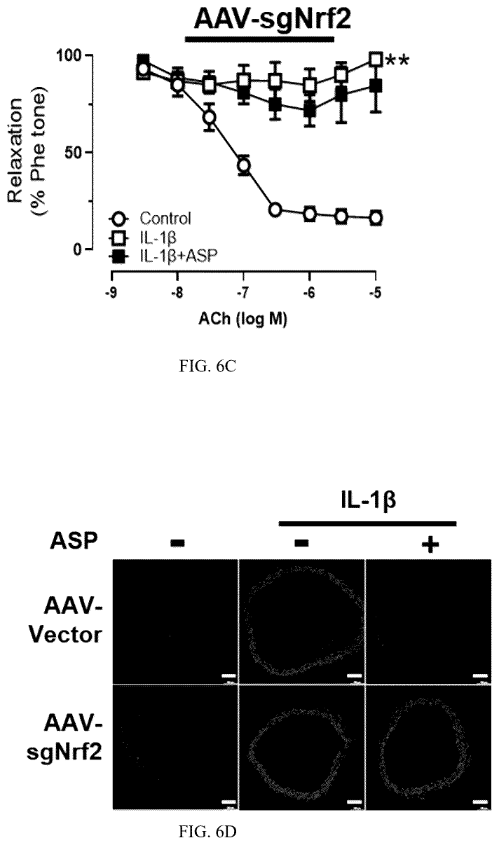

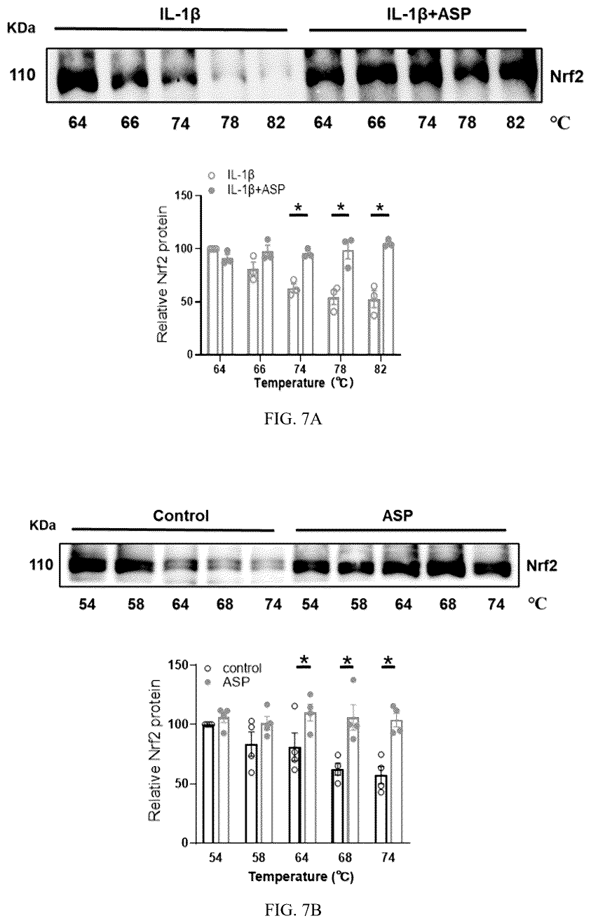

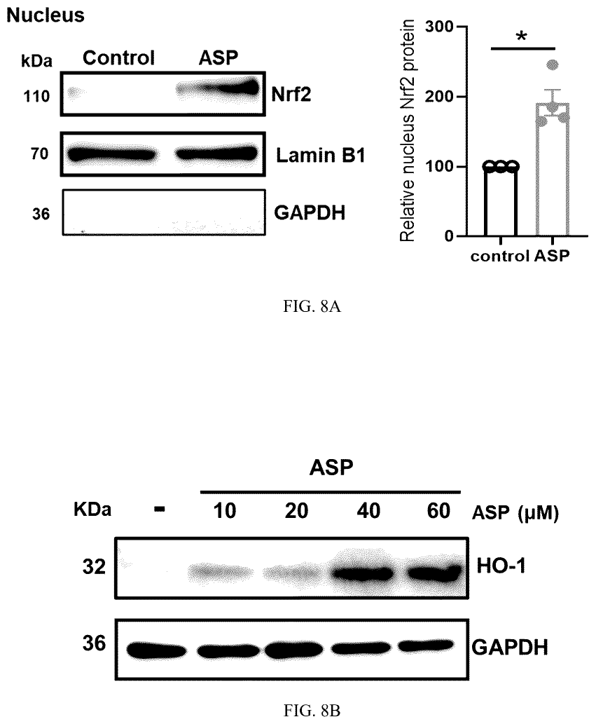

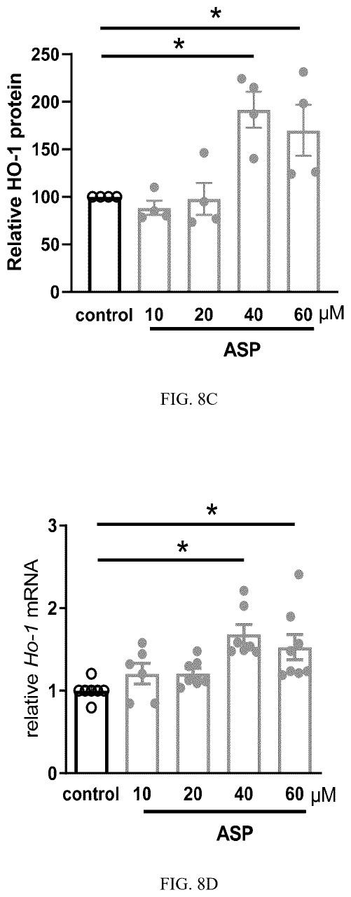

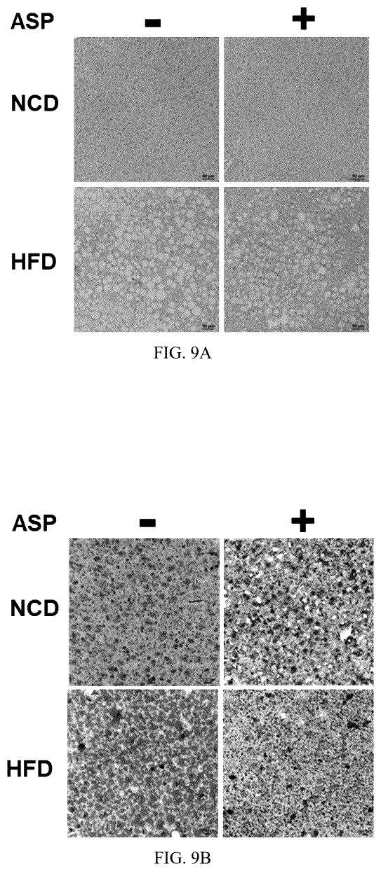

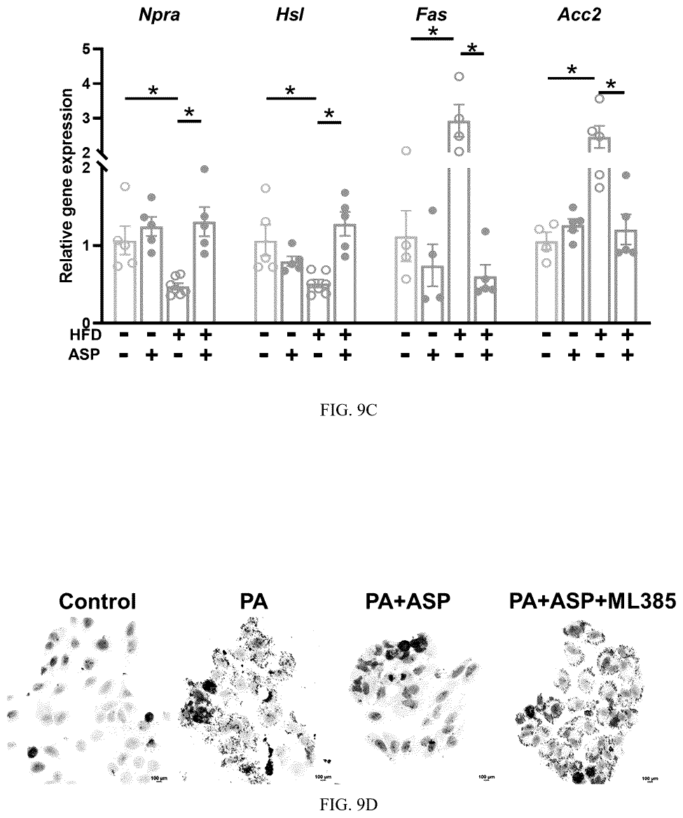

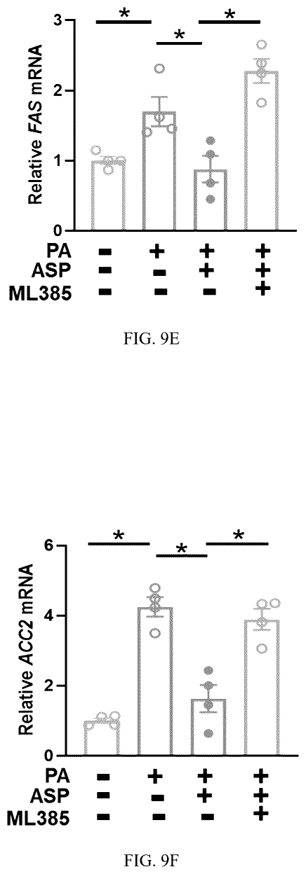

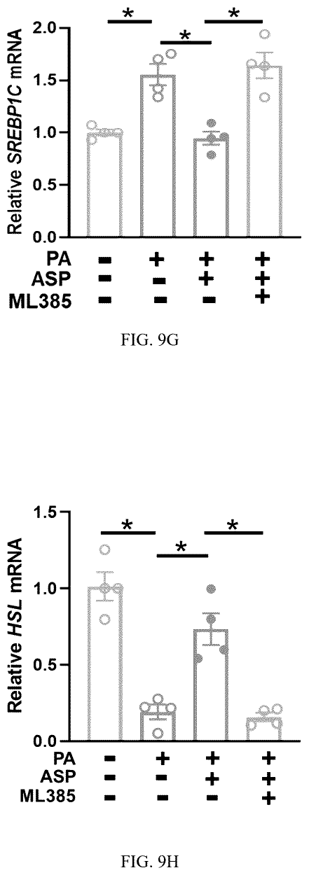

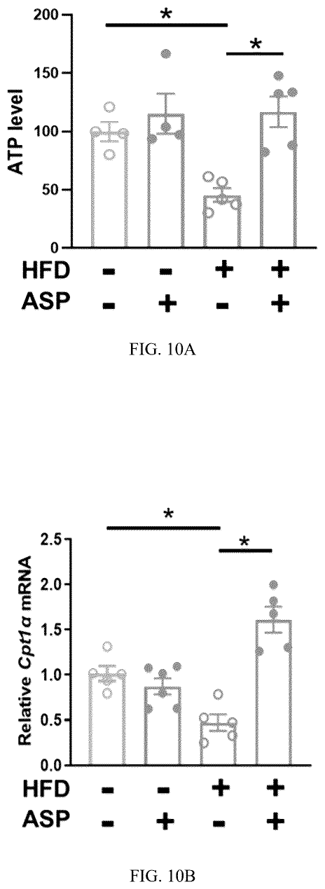

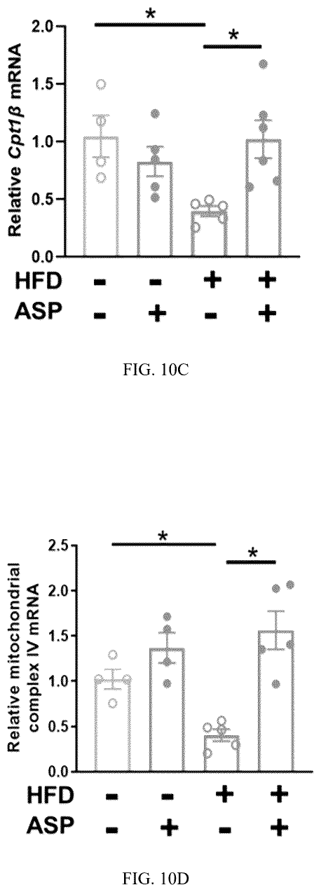

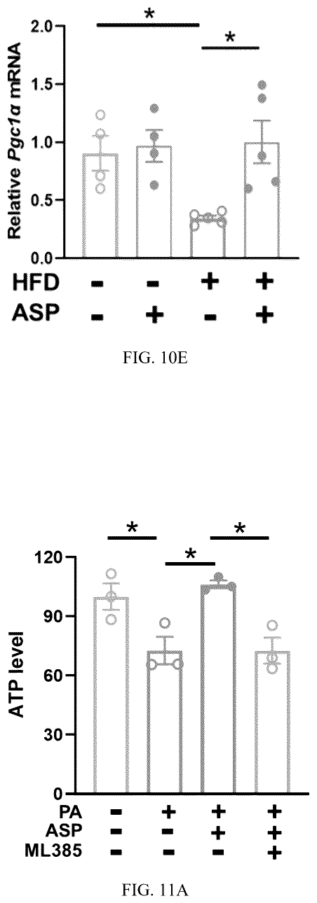

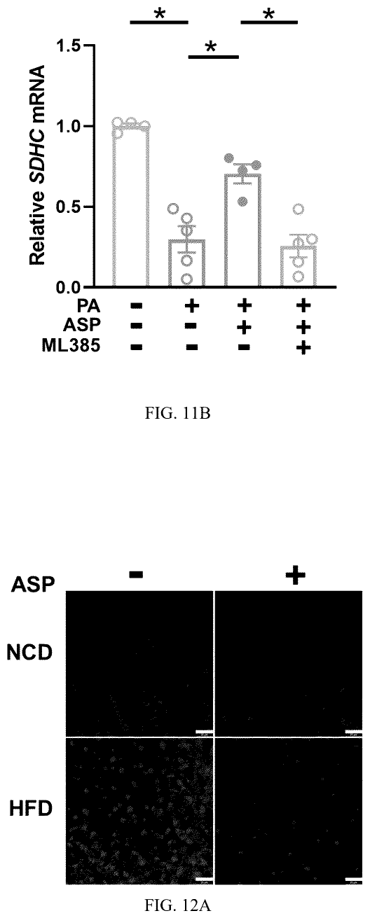

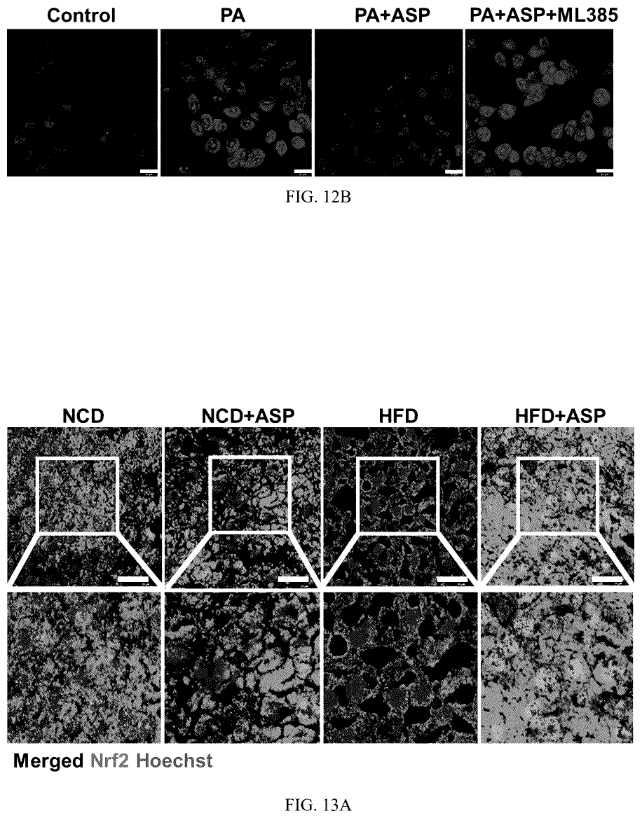

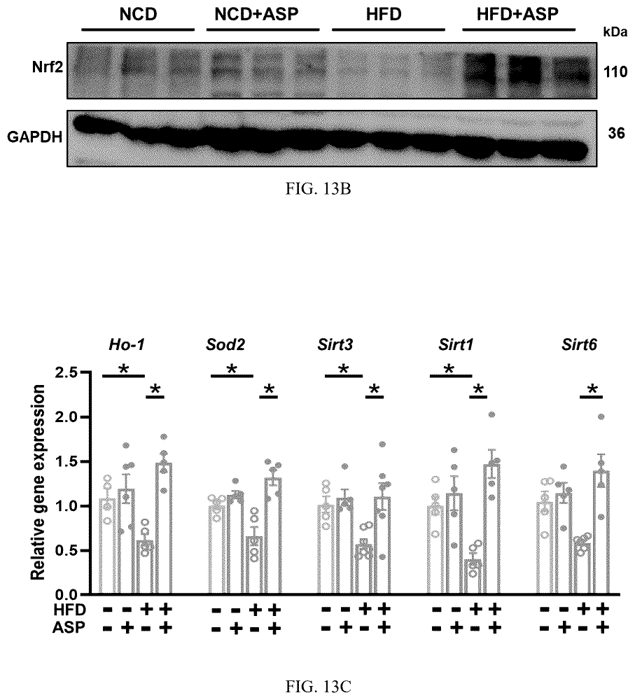

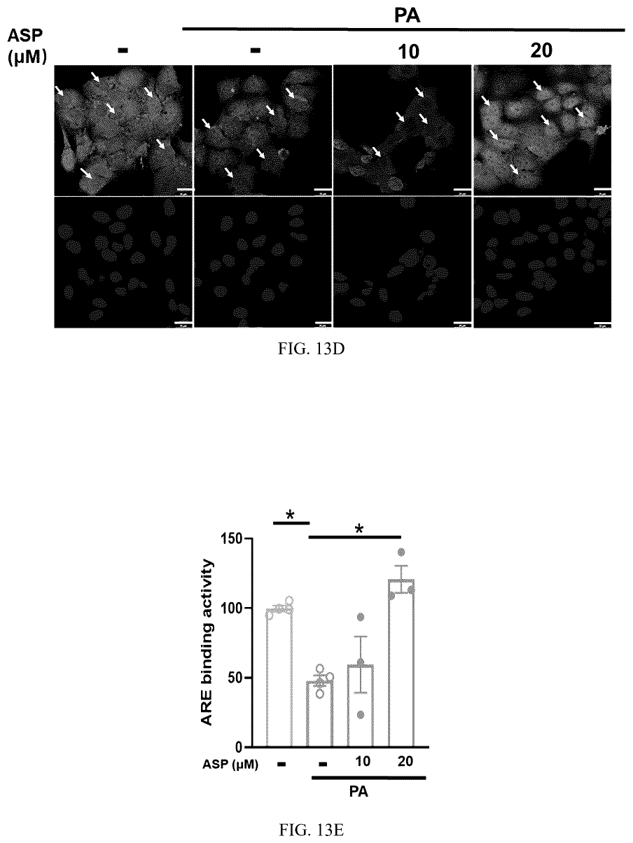

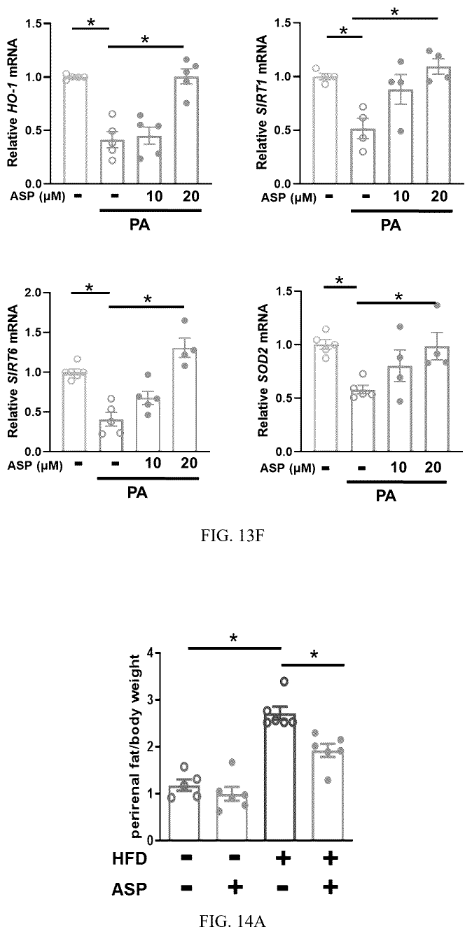

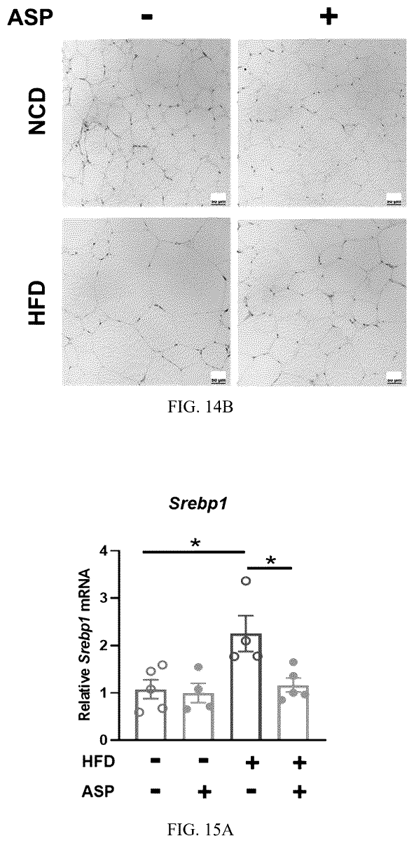

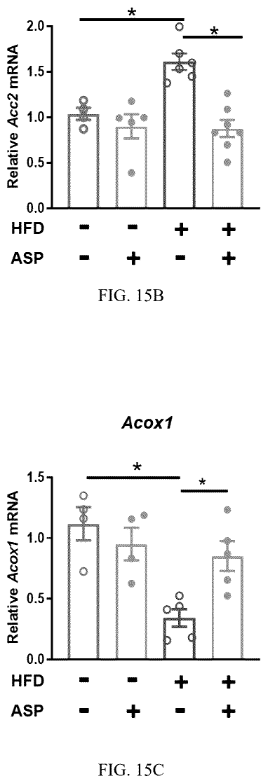

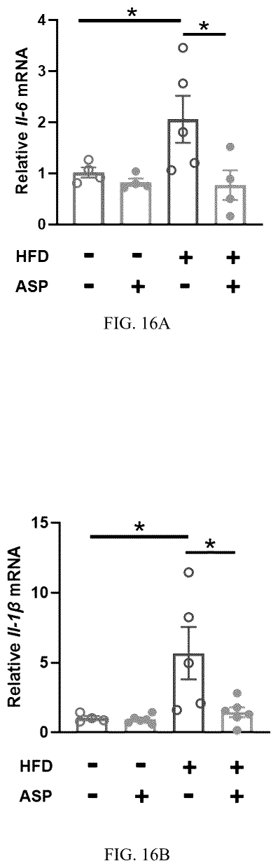

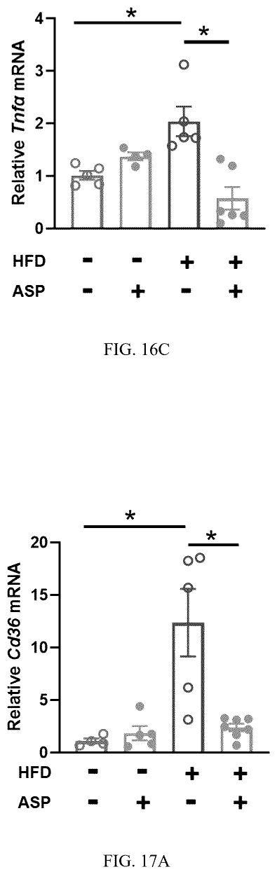

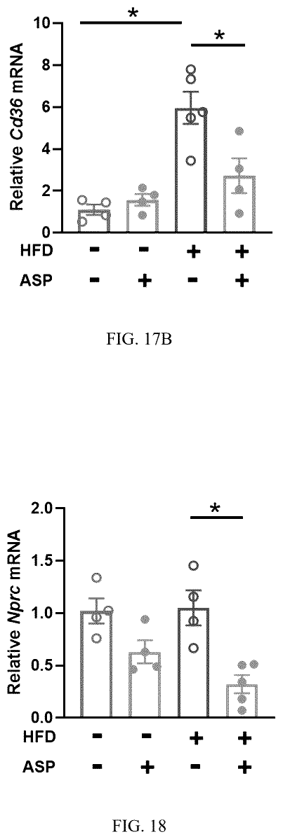

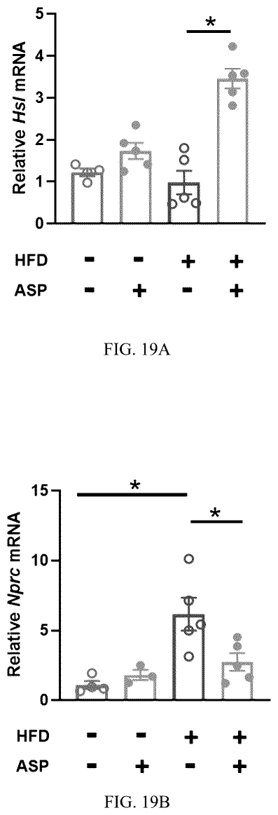

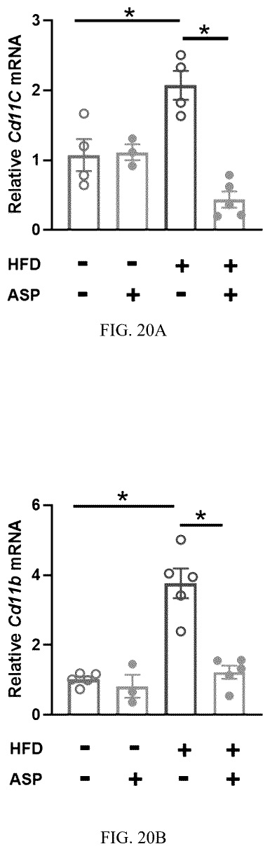

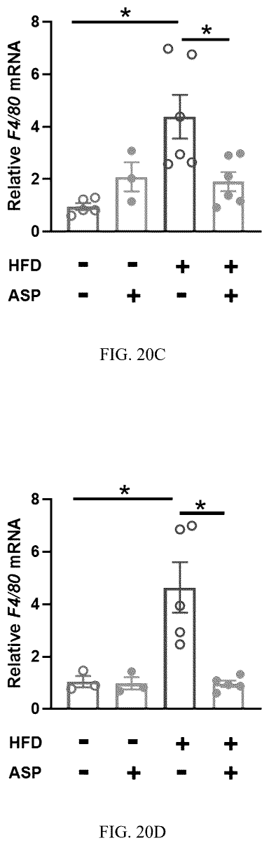

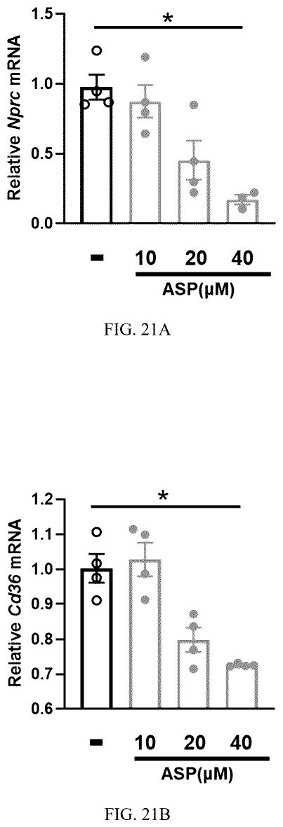

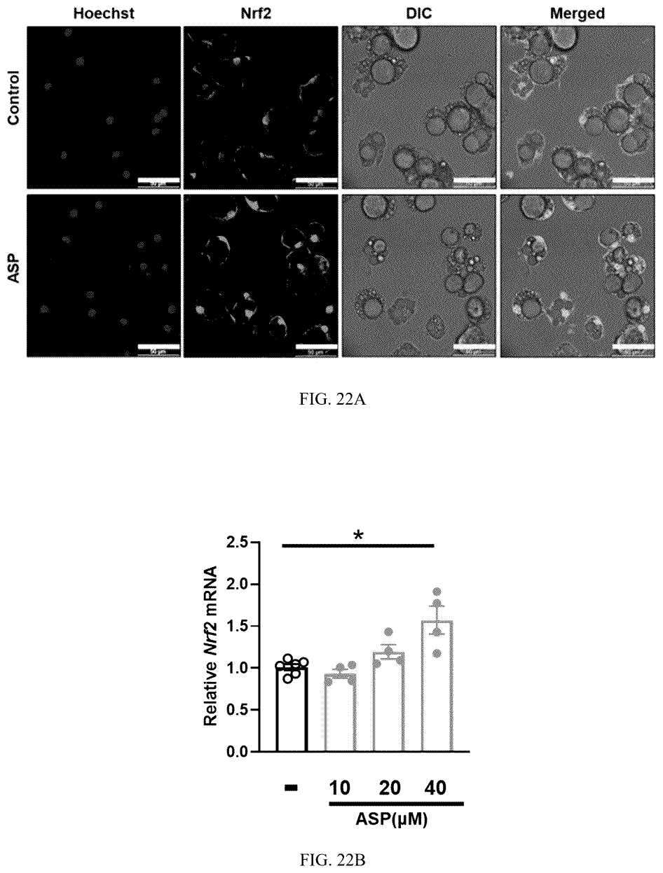

ASP significantly improved endothelial-dependent relaxations (FIGS. 1A-1C), suppressed oxidative stress (FIGS. 1D-1E), reduced VCAM-1 expression by about 2 fold and ICAM-1 expression by about 2 fold (FIG. 1F), increased both Nrf2 expression by about 1.5 fold and HO-1 expression by about 1.5 fold in obese mice aortae (FIGS. 1G-1H). ASP failed to prevent endothelial dysfunction in HO-1 inhibited aortae (FIGS. 2A-2D) and HO-1-siRNA transfected endothelial cells (FIGS. 2E-2F), indicating that ASP improved endothelial function through upregulating HO-1. Nrf2 knockdown attenuated HO-1 upregulation by ASP, showing that ASP regulated Nrf2/HO-1 pathway (FIGS. 3A-3B). ASP increased nuclear Nrf2 (FIG. 3C), and a dual luciferase reporter assay illustrated that ASP increased ARE binding activity (FIG. 3D). Moreover, inhibition of Nrf2/ARE binding by ML385 blocked vascular protective effects and HO-1 upregulation by ASP, showing that ASP activated Nrf2/HO-1 signaling by stimulating Nrf2/ARE binding (FIGS. 3E-3G, FIGS. 4A-4C). ASP rescued endothelial dysfunction and suppressed vascular oxidative stress associated with obesity via activating Nrf2/HO-1 pathway (FIGS. 4D-4F).

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A method of activating the nuclear factor erythroid 2-related factor (Nrf2) signaling pathway in a subject, the method comprising administering an effective amount of Asperuloside to the subject.

Embodiment 2. The method of embodiment 1, wherein the subject has obesity-associated vascular dysfunction, or non-alcoholic fatty liver disease.

Embodiment 3. The method of any of the preceding embodiments, wherein the administration of Asperuloside to the subject fully reverses an impairment of endothelium-dependent relaxation.

Embodiment 4. The method of any of the preceding embodiments, wherein the administration of Asperuloside to the subject decreases expression levels of VCAM-1 and ICAM-1 by about 2 fold.

Embodiment 5. The method of any of the preceding embodiments, wherein the administration of Asperuloside to the subject increases endothelial Nrf2/antioxidant responsive element (ARE) binding in a dose dependent manner to about 50%, 60%, or 70% higher than the binding to impaired endothelial cells when 10 μm ASP, 20 μm ASP, or 40 μm ASP are administered, respectively.

Embodiment 6. The method of any of the preceding embodiments, wherein the Asperuloside binds directly to the Nrf2 protein, wherein the binding increases Nrf2 activity by about 1.5 fold.

Embodiment 7. The method of any of the preceding embodiments, wherein the administration of Asperuloside to the subject rescues endothelial dysfunction and suppresses vascular oxidative stress associated with obesity and IL-1β by activating the Nrf2/HO-1 pathway.

Embodiment 8. The method of any of the preceding embodiments, wherein the administration of Asperuloside to the subject promotes Nrf2 nuclear translocation and activates Nrf2/HO-1 pathway in endothelial cells under basal conditions, wherein Asperuloside protects against impairments of endothelium-dependent relaxations via activating the Nrf2/HO-1 pathway.

Embodiment 9. The method of embodiment 8, wherein the activation of Nrf2/HO-1 pathway increases expression levels of Nrf2 and HO-1 by about 1.5 fold.

Embodiment 10. The method of any of the preceding embodiments, wherein the administration of Asperuloside to the subject stimulates Nrf2 nuclear translocation, wherein the protein level of Nrf2 in the liver increases by about 3 fold or promotes Nrf2 nuclear translocation in palmitic acid impaired HepG2 cells.

Embodiment 11. The method of any of the preceding embodiments, wherein the administration of Asperuloside to the subject promotes Nrf2/ARE binding by about 2 fold or Nrf2 nuclear translocation in palmitic acid-impaired HepG2 cells.

Embodiment 12. The method of any of the preceding embodiments, wherein the administration of Asperuloside to the subject inhibits lipid accumulation by about 50% to 100% by activating Nrf2 signaling.

Embodiment 13. The method of any of the preceding embodiments, wherein the administration of Asperuloside to the subject attenuates lipogenesis, fatty acid uptake in primary adipocytes, inflammation and macrophage infiltration and promotes lipolysis in perineal adipose tissues.

Embodiment 14. The method of embodiment 13, wherein perirenal adipose tissues decreases in weight and sizes by an average of about 35%.

Embodiment 15. The method of any of the preceding embodiments, wherein the administration of Asperuloside to the subject stimulates Nrf2 nuclear translocation and dose-dependently increases by not less than 1.2 fold Nrf2 mRNA expression in primary adipocytes.

Embodiment 16. The method of any of the preceding embodiments, wherein the administration of Asperuloside to the subject fully alleviates mitochondrial dysfunction in the liver by activating hepatic Nrf2 signaling.

Embodiment 17. The method of any of the preceding embodiments, wherein the administration of Asperuloside to the subject increases HO-1 mRNA expression in the liver by about 2.5 fold.

Embodiment 18. The method of any of the preceding embodiments, wherein the administration of Asperuloside to the subject increases HO-1 mRNA expression in palmitic acid-impaired HepG2 cells by at least 2 fold.

Embodiment 19. The method of any of the preceding embodiments, wherein the administration of Asperuloside to the subject attenuates oxidative stress by about 100% and ROS level to approximately basal level in the liver by activating Nrf2 signaling pathway.

Embodiment 20. The method of any of the preceding embodiments, wherein the Asperuloside is administered daily.

Embodiment 21. The method of any of the preceding embodiments, wherein the Asperuloside is at a dose of about 1 mg/kg to about 100 mg/kg.

Embodiment 22. The method of any of the preceding embodiments, further comprising administering Asperuloside with a nonsteroidal anti-inflammatory compound, a calcium channel blocker, a statin, a nitrate, or any combination thereof.

Embodiment 23. The method of embodiment 10, wherein the nonsteroidal anti-inflammatory compound is aspirin, the calcium channel blocker is Amlodipine, Diltiazem, Felodipine, Isradipine, Nicardipine, Nifedipine, Nisoldipine, Verapamil, and the statin is Atorvastatin, Fluvastatin, Lovastatin, Pitavastatin, Pravastatin, Rosuvastatin, or Simvastatin.

We claim:

1. A method of activating the nuclear factor erythroid 2-related factor (Nrf2) signaling pathway in a subject, the method comprising administering an effective amount of Asperuloside to the subject.

2. The method of claim 1, wherein the subject has obesity-associated vascular dysfunction, or non-alcoholic fatty liver disease.

3. The method of claim 1, wherein the administration of Asperuloside to the subject fully reverses an impairment of endothelium-dependent relaxation.

4. The method of claim 1, wherein the administration of Asperuloside to the subject decreases expression levels of VCAM-1 and ICAM-1 by about 2 fold.

5. The method of claim 1, wherein the administration of Asperuloside to the subject increases endothelial Nrf2/antioxidant responsive element (ARE) binding in a dose dependent manner to about 50%, 60%, or 70% higher than the binding to impaired endothelial cells when 10 μm ASP, 20 μm ASP, or 40 μm ASP are administered, respectively.

6. The method of claim 1, wherein the Asperuloside binds directly to the Nrf2 protein, wherein the binding increases Nrf2 activity by about 1.5 fold.

7. The method of claim 1, wherein the administration of Asperuloside to the subject rescues endothelial dysfunction and suppresses vascular oxidative stress associated with obesity and IL-1β by activating the Nrf2/HO-1 pathway.

8. The method of claim 1, wherein the administration of Asperuloside to the subject promotes Nrf2 nuclear translocation and activates Nrf2/HO-1 pathway in endothelial cells under basal conditions, wherein Asperuloside protects against impairments of endothelium-dependent relaxations via activating the Nrf2/HO-1 pathway.

9. The method of claim 8, wherein the activation of the Nrf2/HO-1 pathway increases expression levels of Nrf2 and HO-1 by about 1.5 fold.

10. The method of claim 1, wherein the administration of Asperuloside to the subject stimulates Nrf2 nuclear translocation, wherein the protein level of Nrf2 in the liver increases by about 3 fold or promotes Nrf2 nuclear translocation in palmitic acid-impaired HepG2 cells.

11. The method of claim 1, wherein the administration of Asperuloside to the subject promotes Nrf2/ARE binding by about 2 fold or Nrf2 nuclear translocation in palmitic acid-impaired HepG2 cells.

12. The method of claim 1, wherein the administration of Asperuloside to the subject inhibits lipid accumulation by about 50% to 100% by activating Nrf2 signaling.

13. The method of claim 1, wherein the administration of Asperuloside to the subject attenuates lipogenesis, fatty acid uptake in primary adipocytes, inflammation and macrophage infiltration and promotes lipolysis in perineal adipose tissues.

14. The method of claim 13, wherein perirenal adipose tissues decreases in weight and sizes by an average of about 35%.

15. The method of claim 1, wherein the administration of Asperuloside to the subject stimulates Nrf2 nuclear translocation and dose-dependently increases by not less than 1.2 fold Nrf2 mRNA expression in primary adipocytes.

16. The method of claim 1, wherein the administration of Asperuloside to the subject fully alleviates mitochondrial dysfunction in the liver by activating hepatic Nrf2 signaling.

17. The method of claim 1, wherein the administration of Asperuloside to the subject increases HO-1 mRNA expression in the liver by about 2.5 fold.

18. The method of claim 1, wherein the administration of Asperuloside to the subject increases HO-1 mRNA expression in palmitic acid-impaired HepG2 cells by at least 2 fold.

19. The method of claim 1, wherein the administration of Asperuloside to the subject attenuates oxidative stress by about 100% and ROS level to approximately basal level in the liver by activating Nrf2 signaling pathway.

20. The method of claim 1, wherein the Asperuloside is administered daily.

21. The method of claim 1, wherein the Asperuloside is at a dose of about 1 mg/kg to about 100 mg/kg.

22. The method of claim 1, further comprising administering Asperuloside with a nonsteroidal anti-inflammatory compound, a calcium channel blocker, a statin, a nitrate, or any combination thereof.

23. The method of claim 20, wherein the nonsteroidal anti-inflammatory compound is aspirin, the calcium channel blocker is Amlodipine, Diltiazem, Felodipine, Isradipine, Nicardipine, Nifedipine, Nisoldipine, Verapamil, and the statin is Atorvastatin, Fluvastatin, Lovastatin, Pitavastatin, Pravastatin, Rosuvastatin, or Simvastatin.


SEQUENCE LISTING (4 sequences)

SEQ ID NO: 1 - RNA, 21 nt, synthetic construct
  source 1..21: mol_type = other RNA
gcaggacatggatttgatttt

SEQ ID NO: 2 - RNA, 21 nt, synthetic construct
  source 1..21: mol_type = other RNA
aatcaaatccatgtcctgctg

SEQ ID NO: 3 - RNA, 23 nt, synthetic construct
  source 1..23: mol_type = other RNA
agaaggctttaagctggtgattt

SEQ ID NO: 4 - RNA, 23 nt, synthetic construct
  source 1..23: mol_type = other RNA
atcaccagcttaaagccttcttt